(19) United States
(12) Patent Application Publication
(10) Pub. No.: US 2010/0330557 A1
(43) Pub. Date: Dec. 30, 2010

(54) GENOMIC COORDINATE SYSTEM

(76) Inventors: ZOHAR YAKHINI, Ramat HaSharon (IL); Amir Ben-Dor, Kfar Kava (IL); Brian Jon Peter, Los Altos, CA (US)

Correspondence Address: Agilent Technologies, Inc. in care of:;CPA Global, P. O. Box 52050, Minneapolis, MN, 55402, US

(21) Appl. No.: 12/495,541

(22) Filed: Jun. 30, 2009

(51) Int. Cl. C12Q 1/68; G01N 33/50

(52) U.S. Cl. 435/6; 702/19

(57) ABSTRACT

A method of sample analysis is provided. In certain embodiments, the method comprises: a) site-specifically labeling a test genome with at least two different labels to produce a labeled genome labeled at a plurality of discrete sites across the genome; b) stretching a nucleic acid of the labeled genome to produce a linear pattern of the different labels along a region of a stretched nucleic acid; c) reading the labels along the region to provide a test pattern comprising a sequence of colors emitted by the labels; d) comparing the test pattern to a plurality of reference patterns obtained from a reference genome, in which the reference patterns are mapped to corresponding genomic locations in the reference genome; and e) identifying one or more reference patterns that match the test pattern, thereby mapping a location for the region in the test genome.

### INTRODUCTION

In spite of the advent of new sequencing technologies, there remains a need to analyze the “connectivity” of DNA segments. Most sequencing, PCR, and array-based approaches analyze a single, small fragment of the genome. These approaches are unable to easily measure structural rearrangements such as translocations and inversions. Copy number variations can be measured, but are difficult to map onto a genomic context. Conversely, cytogenetic technologies such as FISH and karyotyping are generally either low-resolution or too focused to be useful on a genomic scale. There are no technologies, currently, that allow for robust and accurate determinations of translocations and haplotypes in a genomic scale. As translocations play a major role in cancer pathogenesis and in tumor characterization, this shortcoming of current technology is rather limiting. Therefore, there remains a need for technologies that identify sequences on a chromosomal scale, which are then used for mapping other measured events.

This disclosure relates in part to a method of genome analysis using a coordinate system to identify locations in the genome.

### SUMMARY

A method of sample analysis is provided. In certain embodiments, the method comprises: a) site-specifically labeling a test genome with at least two different labels to produce a labeled genome labeled at a plurality of discrete sites across the genome; b) stretching a nucleic acid of the labeled genome to produce a linear pattern of the different labels along a region of a stretched nucleic acid; c) reading the labels along the region to provide a test pattern comprising a sequence of colors emitted by the labels; d) comparing the test pattern to a plurality of reference patterns obtained from a reference genome, in which the reference patterns are mapped to corresponding genomic locations in the reference genome; and e) identifying one or more reference patterns that match the test pattern, thereby determining, e.g. mapping, a location for the region in the test genome.

### BRIEF DESCRIPTION OF THE FIGURES

FIG. 1 schematically illustrates an embodiment of the method described herein.

FIG. 2 schematically illustrates certain features of some embodiments of the method described herein.

FIG. 3 depicts the percentage of uniquely tagged fragments based on an in silico experiment carried out for a resolution of 2 kbp, 5 kbp, and 10 kbp.

### DEFINITIONS

The term “sample”, as used herein, relates to a material or mixture of materials, typically, although not necessarily, in liquid form, containing one or more analytes of interest.

The term “genome”, as used herein, relates to a material or mixture of materials, containing genetic material from an organism. The term “genomic DNA” as used herein refers to deoxyribonucleic acids that are obtained from an organism (e.g. cultured cell lines). The terms “genome” and “genomic DNA” encompass genetic material that may have undergone amplification, purification, or fragmentation. The term “test genome,” as used herein refers to genomic DNA that is of interest in a study. A genome may encompass the entirety of the genetic material from an organism, or it may encompass only a selected fraction thereof: for example, the test genome may encompass one chromosome from an organism with a plurality of chromosomes.

The term “reference genome”, as used herein, refers to a sample comprising genomic DNA to which a test sample may be compared. In certain cases, reference genome contains regions of known sequence information.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten, fluorescent labels, or radiolabels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes. Nucleotides may include those that when incorporated into an extending strand of a nucleic acid enables continued extension (non-chain terminating nucleotides) and those that prevent subsequent extension (e.g. chain terminators).

The term “chain terminator” or “chain terminator nucleotide”, as used herein, denotes a nucleotide as defined above but with certain modifications to prevent nucleic acid extension from the chain terminator nucleotide. Stated differently, a chain terminator is derived from a monomeric unit of nucleic acid polymers but is modified such that they prevent subsequent polymerization. One example of a chain terminator is dideoxynucleotide.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “oligonucleotide”, as used herein, denotes a single-stranded multimer of nucleotides from about 2 to 500 nucleotides, e.g., 2 to 200 nucleotides. Oligonucleotides may be synthetic or may be made enzymatically, and, in some embodiments, are under 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150 or 150 to 200, up to 500 or more nucleotides in length, for example.

The term “duplex” or “double-stranded” as used herein refers to nucleic acids formed by hybridization of two single strands of nucleic acids containing complementary sequences. In most cases, genomic DNA are double-stranded.

The term “probe”, as used herein, refers to a nucleic acid that is complementary to a nucleotide sequence of interest. In certain cases, detection of a target analyte requires hybridization of a probe to a target. In certain embodiments, a probe may be immobilized on a surface of a substrate. A “substrate” can have a variety of configurations and material, e.g., a sheet, bead, glass cover slip, or other structure. In certain embodiments, a probe may be present on a surface of a planar support, e.g., in the form of an array.

The terms “determining”, “measuring”, “evaluating”, “assessing”, “analyzing”, and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

As used herein, the term “single nucleotide polymorphism”, or “SNP” for short, refers to single nucleotide position in a genomic sequence for which two or more alternative alleles are present at appreciable frequency (e.g., at least 1%) in a population.

The term “region” or “chromosomal region”, as used herein, denotes a contiguous length of nucleotides in a genome of an organism. A chromosomal region may be in the range of 1000 nucleotides in length to an entire chromosome, e.g., 100 Kb to 10 Mb for example.

The term “sequence alteration”, as used herein, refers to a difference in nucleic acid sequence between a test sample and a reference sample that may vary over a range of 1 to 10 bases, 10 to 100 bases, 100 to 100 Kb, or 100 Kb up to 10 Mb, or more. Sequence alteration may include single nucleotide polymorphism and genetic mutations relative to wild-type. Sequence alteration encompasses “chromosomal rearrangement” that results from one or more parts of a chromosome being rearranged within a single chromosome or between chromosomes relative to a reference. In certain cases, a sequence alteration may reflect an abnormality in chromosome structure, such as an inversion, a deletion, an insertion or a translocation, for example.

As used herein, the term “data” refers to a collection of organized information, generally derived from results of experiments in lab or in silico, other data available to one of skilled in the art, or a set of premises. Data may be in the form of numbers, words, annotations, or images, as measurements or observations of a set of variables. Data can be stored in various forms of electronic media as well as obtained from auxiliary databases

The term “stretching”, as used herein, refers to the act of elongating a DNA molecule so to minimize the amount of tertiary structures, e.g. unfolding coiled DNA structures.

The term “homozygous” denotes a genetic condition in which identical alleles reside at the same loci on homologous chromosomes. In contrast, “heterozygous” denotes a genetic condition in which different alleles reside at the same loci on homologous chromosomes.

The term “color” is an arbitrarily assigned descriptor for a particular label, which is distinguishable from labels of other colors. A color may correspond to an emission spectrum, where different emission spectra from distinguishable labels correspond to different descriptors. In certain cases, the descriptor assigned to an emission spectrum emitted by a label may be a color, e.g., “red” or “green”, that corresponds to a region of the visible light spectrum containing the wavelength at which the emission spectrum reaches a maximum. In certain cases, the labels may be distinguished by size, e.g., colloidal gold particles of 5 nm diameter versus colloidal gold particles of 15 nm. In this case the distinguishable labels may be arbitrarily assigned different colors, e.g., 5 nm as “red” and 15 nm gold as “green”, or the particles may be rendered in an image in different colors.

The term “label” refers to a molecule or tag which is detectable by an imaging system. Exemplary labels include fluorescent molecules such as cyanine dyes (e.g., Cy3 and Cy5), fluorescent proteins such as green fluorescent protein, haptens such as biotin, and the like. Labels may be selected from the group comprising fluorescent dyes, chemiluminescent molecules, chromogenic substrates, radioisotopes, colloidal gold particles, enzyme substrates, biotin, molecules exhibiting detectable nuclear magnetic resonance, seminconductor nanocrystals, proteins, peptides, antibodies, carbohydrates, and lipids.

The term “imaging” refers not only to the collection of data in visible wavelengths (e.g., light microscopy), but also to the collection of wavelengths not visible to the naked eye, e.g., infrared or ultraviolet wavelengths, or the collection of electrons, e.g., electron microscopy. Furthermore, imaging may refer to the collection of data in a form other than light, e.g., surface topography measurements collected by atomic force microscopy, which are then rendered as an image with the aid of a computer. Data collection systems suitable for imaging may include light microscopes, atomic force microscopes, transmission electron microscopes, scanning tunneling microscopes, near-field detection systems, total internal reflection microscopes, and the like.

As used herein, the term “linear pattern” refers to a pattern of labels that is generated in an image when labels at a plurality of sites across a stretched region of a genome are visualized. The linear pattern in an image is derived from wavelengths of the spectrum peak emitted by the labels (e.g. colors) and/or spatial components (e.g. distance between labels) collected as data by a detection apparatus (e.g. a microscope). In certain embodiments, a linear pattern is a contiguous sequence of “colors” in an order of their positions along a contiguous stretched region of a genome.

A “distinct pattern” or “distinctly labeled”, as used herein, refers to a linear pattern of a region of a labeled nucleic acid that is different from all other regions of nucleic acids in the genomic sample of interest and identifies the region out of all other regions in the sample. A certain level of complexity is required in a distinct pattern depending on the length of the region that needs to be uniquely identified out of the total number of regions in the sample.

The term “reference pattern”, as used herein, refers to a pattern generated in an image when labeled nucleotides incorporated into a known nucleic acid sequence of a reference genome are visualized. The reference pattern may be derived from experiments or from calculations in silico. In certain cases, the reference genome is the same species as that of the genomic sample of interest.

As used herein, “test pattern” refers to an information string representing a linear pattern of a stretched nucleic acid. Information conveyed by a test pattern encompasses one or more of the following: sequential order of labels, type of labels, color emitted by labels, location of labels, and distance between labels on a region of a stretched nucleic acid. In certain cases, the test pattern encompasses information regarding the color emitted labels and the sequential order at which those labels are located in order of their positions along a region of the stretched nucleic acid. An exemplary test pattern may be green, green, red, green (GGRG).

As used herein, “mapping” refers to the process of identifying a region of a test genome as the same as a specific region of a reference genome, and consequently, provides the genomic context of the region of the test genome. The genomic context denotes a location or address of the region in the genome, e.g. chromosome 9, cytoband q10. Ranges for the start and end positions may be used to provide genomic context, e.g. chromosome 8, start position 13452517, end position 15721630. In other words, mapping provides a location for the region of the test genome by correlating a test pattern derived from the region of the test genome with one or more reference patterns.

### DESCRIPTION OF EXEMPLARY EMBODIMENTS

A method of sample analysis is provided. In certain embodiments, the method comprises: a) site-specifically labeling a test genome with at least two different labels to produce a labeled genome labeled at a plurality of discrete sites across the genome; b) stretching a nucleic acid of the labeled genome to produce a linear pattern of the different labels along a region of a stretched nucleic acid; c) reading the labels along the region to provide a test pattern comprising a sequence of colors emitted by the labels; d) comparing the test pattern to a plurality of reference patterns obtained from a reference genome, in which the reference patterns are mapped to corresponding genomic locations in the reference genome; and e) identifying one or more reference patterns that match the test pattern, thereby mapping a location for the region in the test genome.

Before the present invention is described in greater detail, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Method of Genome Analysis

A method of sample analysis is provided. In certain embodiments, the method comprises: a) site-specifically labeling a test genome with at least two different labels to produce a labeled genome labeled at a plurality of discrete sites across the genome; b) stretching a nucleic acid of the labeled genome to produce a linear pattern of the different labels along a region of a stretched nucleic acid; c) reading the labels along the region to provide a test pattern comprising a sequence of colors emitted by the labels; d) comparing the test pattern to a plurality of reference patterns obtained from a reference genome, in which the reference patterns are mapped to corresponding genomic locations in the reference genome; and e) identifying one or more reference patterns that match the test pattern, thereby determining, e.g. mapping, a location for the region in the test genome.

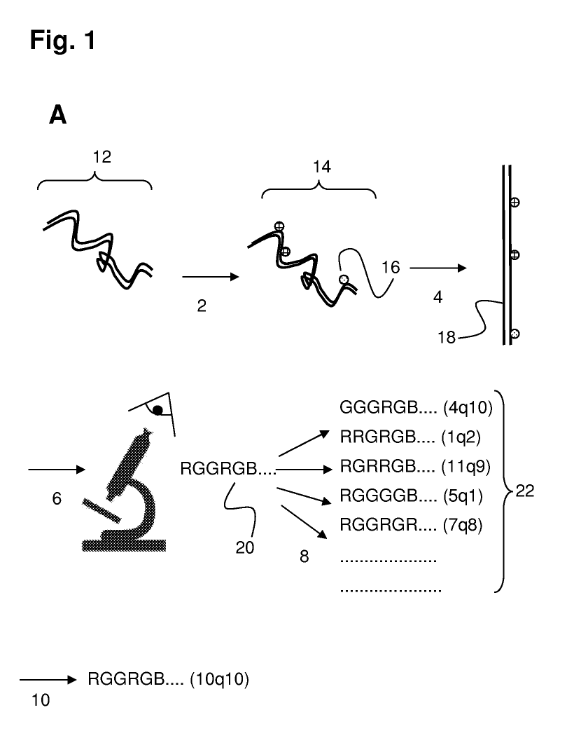

Certain features of the subject method are illustrated in FIG. 1 and are described in greater detail below. With reference to FIG. 1, the method involves labeling 2 test genome 12 in a sequence specific manner with at least two different labels to produce labeled genome 14. The labels incorporated into the genome, one of which is shown as label 16, may be distributed across the genome at a plurality of discrete sites. The labeled test genome is then stretched 4 so that a region of the labeled test genome is elongated to remove tertiary structures. The next step involves reading 6 the linear pattern of labels on the stretched labeled region 18 of the test genome to provide test pattern 20. The reading step 6 converts information regarding the colors of labels and the sequential order of labels along a contiguous stretched region and converting that information to a test pattern. Other information may also be included in the test pattern. The test pattern is then compared 8 to a plurality of reference patterns 22. Each of the plurality of reference patterns represents a known region of a reference genome. If the test pattern is found to be the same as one or more of the reference patterns, the found match between the test pattern and the reference pattern indicates that the region of the test genome has the same genomic location as that of the known region of the reference genome. This match allows one or more genomic locations to be mapped to the genomic region of interest. For example, if the test pattern matches a reference pattern that corresponds to chromosome 10, cytoband q10, the region of the test genome is identified in step 10 to be cytoband q10 of chromosome 10.

As mentioned above, the test pattern represents a sequence of colors and optionally distances therebetween where a color as defined previously, is an arbitrarily assigned descriptor for an emission spectrum, where different emission spectra from distinguishable labels may correspond to different descriptors. For example, certain labels having a maximum emission peak at about 510 nm may be referred to as green labels. Some sites could be labeled with two or more colors in such close proximity as to give a third color distinguishable from the original colors in the combination. In such embodiments, the third color may be described as having the combination of the wavelengths at which two or more emission spectrums of two or more different labels reach their maxima. For example, a site labeled with both green and red may yield yellow as the color of the labeled site.

As shown in FIG. 1, the labeling step 2 may be performed by contacting a sample comprising test genome 12 with reagents for incorporating labels. In certain cases, the test genome may be fragmented by sonication or nebulization (e.g. into sizes between 10 Kb up to 10,000 Kb or more), amplified, or partially purified prior to the contacting step 2. In other cases, the sample may comprise a partitioned genome, e.g., a genome that has had material subtracted from it. The test genome may also be enzymatically treated prior to contacting step 2. Any of the known means for labeling nucleic acid may be used to label the test genome in labeling step 2 of the subject method. Certain exemplary site-specific labeling methods are discussed below.

Site-specifically labeling a test genome may encompass contacting the test genome with labeled oligonucleotides under hybridization conditions. The test genome may be contacted with a plurality of oligonucleotides so that the oligonucleotides would hybridize across the genome in discrete locations. Hybridization conditions are designed to promote specific binding of oligonucleotides to complementary nucleotide sequences on the test genome. The hybridization conditions may vary depending on the length and composition of the region of complementarity between the oligonucleotides and their respective target sites on the test genome. Suitable conditions are nevertheless known and described in, e.g., Sambrook et al, supra. In certain cases, conditions suitable for successful hybridization of an oligonucleotide and a complementary region of the test genome may be determined by calculating the Tₘ of the expected oligonucleotide duplex in a particular hybridization buffer using the formula Tₘ=81.5+16.6(log₁₀[Na⁺])+0.41 (fraction G+C)— (60/N), where N is the chain length and [Na⁺] is less than 1 M. Suitable hybridization conditions may also be determined experimentally.

The oligonucleotides may be designed to be complementary to specific sequences in the test genome and may be, e.g., 10 to 20, 11 to 30, 31 to 40, 41 to 50, 51-60, 61 to 70, 71 to 80, 80 to 100, 100 to 150, 150-300, up to 500 or more base pairs in length. The detectable label on each oligonucleotide may comprise one or more tags that emits one or more colors or a non-fluorescent tag that is further processed for visualization. Each oligonucleotide hybridized to a site would contribute one unit of information to the test pattern (e.g. red). Two oligonucleotides hybridized to two labeled sites may contribute two colors and/or a distance between the two sites in a test pattern. As mentioned above, if one site is labeled with two or more tags that emit emission peaks at different wavelengths, the color read from this labeled site may represent an average of the multiple wavelengths. Accordingly, one oligonucleotide hybridized to a labeled site does not constitute a sequence of colors but a sequence of colors comprises at least two labeled sites (e.g. two oligonucleotides hybridized to two sites). Some oligonucleotides in the plurality may have the same nucleotide sequence, same length, and the same type of detectable labels. Other oligonucleotides may have different nucleotide sequence but may or may not have the same type of labels. Various combinations of oligonucleotide sequences and their label types may be designed to achieve specific linear patterns for the genomic regions of interest.

In certain embodiments, the oligonucleotide used to site-specifically label the test genome may be designed to hybridize to a region of the test genome in such a way that when hybridized, the backbone of the oligonucleotide runs parallel to the backbone of the complementary target region in the test genome. In such an oligonucleotide, there may be about at least 50%, 60%, 70%, or 90% complementarity between the oligonucleotide and the region of the test genome. In certain cases, an oligonucleotide hybridizes to a region of the test genome with a 100% complementarity.

In alternative embodiments, the oligonucleotide is designed to be a padlock probe, such as a molecular inversion probe, as described in Cao et al. Trends in Biotechnology (2004) 22:38-44. In certain cases, the oligonucleotide may also hybridize to a region of the test genome at its 3′ and 5′ ends and forms a structure like a Q probe as described in a copending application Ser. No. 12/264,091. In certain cases, the oligonucleotide may specifically bind to a duplex nucleic acid of the test genome to produce a triplex, as described in Knauert et al. Human Molecular Genetics (2001) 10:2243-2251.

The oligonucleotide may contain modifications that allow for stringent hybridization and/or strand invasion. Modifications encompass having one or more nucleotides or monomers that are not naturally-occurring in the oligonucleotide. Some exemplary modified monomers include locked and unlocked nucleic acids, a peptide nucleic acid (PNA), a bis PNA clamp, a pseudocomplementary PNA, a co-polymers of the above such as DNA-LNA co-polymers. See, for example, Jensen et al. Nucleic Acids Symposium Series (2008) 52:133-134, Koshkin et al. Tetrahedron (1998) 54, 3607-3630, and U.S. Pat. No. 6,794,499, the disclosure of which is incorporated herein by reference. In certain cases, the oligonucleotides may be coated with proteins, small molecules or enzymes such as recombinase RecA, as described in Rice et al. Genome Res. (2004) 14:116-125.

In other cases, site-specifically labeling a test genome involves one or more agents (e.g. enzymes), such as nicking endonucleases that nick backbones of nucleic acids, polymerases that incorporate one or more nucleotides, transferases that transfer one or more functional groups on to a nucleotide (e.g. methyltransferase), small molecules, antibodies, etc., in a sequence specific manner.

One way to label a test genome is to use a nicking endonuclease and a polymerase to incorporate a labeled nucleotide. Conditions and reagents suitable for the nicking activity of site-specific nicking endonucleases are known to one of skilled in the art. Exemplary methods and experimental conditions suitable for an active site-specific nicking endonucleases and for subsequent nucleotide incorporation may be found in Jo K et al. (PNAS 104:2673-2678, 2007) and Xiao M et al. (Nucleic Acids Res. 35:e16, 2007), and are described in a copending patent application attorney docket no. 20080439.

An alternative way to label a test genome is to contact a site-specific methyltransferase with a test genome. The methyltransferase in the presence of a cofactor transfers either a functional group or a functional group conjugated to a detectable label to an acceptor nucleotide in the test genome. For example, the C5 carbon of cytosine, N4 nitrogen of cytosine, or N6 nitrogen of adenine may be labeled by a methyltransferase. Details of employing a site-specific methyltransferase to label a nucleic acid may be found in a copending patent application Ser. No. 12/325,562.

One other way of performing the step of site-specifically labeling a test genome is to employ DNA binding proteins, such as zinc finger proteins. These proteins may bind to specific transcription factor binding sites but may also be engineered to bind to other nucleotide sequences of choice. The proteins may be tagged with a fluorescent label for visualization or other types of detectable label for processing. A DNA binding protein that may be used in the subject method encompasses various ZFPs developed by Sangamo Biosciences® Inc.

Another example of how one may site-specifically label a test genome is to employ antibodies that have specific affinity for one or more nucleotide sequences in the test genome. Similarly to the DNA binding proteins described above, the antibodies may comprise fluorescent labels or they may be visualized upon contacting with secondary antibodies. Antibodies used herein encompass polyclonal and monoclonal antibody preparations where the antibody may be of any class of interest (e.g., IgM, IgG, and subclasses thereof), as well as preparations including hybrid antibodies, altered antibodies, F(ab′)₂ fragments, F(ab) molecules, Fv fragments, single chain fragment variable displayed on phage (scFv), single chain antibodies, single domain antibodies, diabodies, chimeric antibodies, humanized antibodies, and functional fragments thereof which exhibit immunological binding properties of the parent antibody molecule.

In certain embodiments, the subject method may employ more than one way of labeling a test genome in a sequence-specific manner. For example, the test genome may be contacted with labeled oligonucleotides and labeled antibodies in order to produce a labeled genome. The labeling step may also employ more than one agents simultaneously or sequentially. Various combinations of the labeling means described above are envisioned herein.

In any of the labeling means employed in the subject method, the test genome is labeled with at least two different labels. Where the test genome is hybridized to a plurality of oligonucleotides, each of at least two oligonucleotides that are different in nucleotide sequence may have a different label. For example, an oligonucleotide with a first nucleotide sequence may be tagged with a green fluorescent label while another oligonucleotide with a second nucleotide sequence is tagged with a red fluorescent label. Where the test genome is contacted with one or more agents that incorporate labels or labeled nucleotides onto the test genome, the labeling is performed in the presence of two different labels in the same time or sequentially. For example, one agent that recognizes a specific nucleotide sequence in the test genome is contacted with the test genome in the presence of a green label. The test genome is then contacted with a second agent that recognizes a different nucleotide sequence from the first agent in the presence of a red label. As such, the green and red labels are incorporated into the test genome in a site-specific manner and each color represents a different nucleotide sequence at the site of incorporation. Similarly, agents such as enzymes that incorporate labeled nucleotides may be contacted with the test genome in the presence of two or more different types of labeled nucleotides, e.g. red-adenines and green-thymines. The protocols may be carried out in numerous ways known in the art as long as each type of incorporated label conveys information relating to the nucleotide sequence at the location of the label and/or the method by which the label is incorporated. In light of the many labeling protocols routinely practiced, one of ordinary skill in the art would know how to employ the various reagents associated with labeling a nucleic acid and to design specific protocol to allow a test genome to be labeled with at least two different labels.

Referring to FIG. 1, labeling step 2 may be carried out in vitro or in situ. Cell extracts and tissue preparations may be utilized in these contacting steps. All steps of an in vitro labeling method may also be performed in a single tube. In other cases, steps may be performed on a substrate. For example, the substrate genome may be immobilized onto a bead or a planar surface. Accordingly, after the site-specifically labeling step, the test genome comprise multi-color labels, in which there may be at least two, three, four or more types of labels at a plurality of discrete sites across the genome. In some cases, the density of labeled nucleotides incorporated into a region of a double-stranded DNA may be no more than about once every 1000 bp, 2000 bp, 5 Kb, or 10 Kb, such that the distance between labels is resolvable by a light microscope. In certain cases, the distance between labels is at least near or above the diffraction limit for visible wavelengths of light.

In certain cases, the double-stranded DNA under study is stained with a nonspecific label, such as an intercalating fluorescent dye or other dyes that would label DNA in a non-sequence specific manner (e.g. DAPI, Hoechst, YOYO-1, YO-PRO-1, or PicoGreen). In related embodiments, a labeled site may participate in fluorescence energy transfer (FRET) with an adjacent labeled site or with the stained DNA backbone. The FRET signal is then imaged the same way as the embodiments described above to generate a pattern of labeled sites in order of positions along a contiguous length of the stretched double-stranded DNA.

After the test genome are labeled, the labeled test genome 14 as shown in FIG. 1 is stretched out 4 to provide a linear pattern of labels along a stretched labeled nucleic acid 18. Many ways for stretching nucleic acid including the stretching devices used therein are known in the art. In certain cases, the labeled genome is stretched out into a linear or close to linear form in order to detect the labels on the DNA. Double-stranded DNA in aqueous solutions usually assumes a random-coil conformation. Similar to the method used in Fiber-FISH, the labeled genome comprising coiled DNA molecules may be unwound and stretched into a linear form on a modified glass surface and individually imaged by microscopy, e.g. confocal, epifluorescence, internal reflection fluorescence. Briefly, the method may involve the following steps. First, the DNA is pipetted onto the edge of a glass slide. The solution comprising the DNA is then drawn under the coverslip by capillary action, causing the DNA molecules of the genome to be stretched and aligned on the coverslip surface. As a result, an array of combed single DNA molecules is prepared by stretching molecules attached by their extremities to a glass surface with a receding air-water meniscus. This method is also referred to as molecular combing. By detecting the labels on the combed DNA, labels may be directly visualized, providing a means to construct physical maps and to detect micro-rearrangements. Details of a method using microscopy to detect stretched genomic DNA may be found in Xiao M et al. (2007) “Rapid DNA Mapping by fluorescent single molecule detection” Nucleic Acids Res. 35:e16.

In other embodiments, the DNA molecules of the genome may be stretched 4 as they flow through a microfluidic channel. The hydrodynamic forces in a microfluidic channel generated in laminar flow help to uncoil and to stretch the DNA molecules as they travel with the flow. The solution is pressure driven to provide a flow acceleration over a distance comparable to the size of the DNA molecule. In this approach, a stretched DNA molecule travels through posts of focused light to excite a fluorophore label, for example. The label is detected as the DNA molecules pass through the detectors placed appropriately to capture the signal emitting from the microchannel. Details of using microfluidic channel to stretch and analyze single molecules may be found in US Pat Pub 20080239304 and 20080213912, disclosures of the patent publications are incorporated herein by reference.

In alternative embodiments, the DNA molecules of the genome may be stretched as they flow through a nanofluidic channel. In these embodiments, the nanofluidic channel may have a diameter of less than 200 nm, for example, less than 150 nm, less than 100 nm, less than 50 nm, or less than 20 nm. The confinement of the DNA molecules in the nanochannels leads to elongation of the DNA molecules, allowing optical interrogation. See e.g., Tegenfeldt et al (2004) Proc. Nat. Acad. Sci. USA 101:10979-10983; and Douville et al. Anal. Bioanal. Chem. 391:2395-2409, 2008.

After the stretching step 4, the linear pattern of labels on the stretched nucleic acid is then read 6 to provide test pattern 20. The reading step 6 may encompass imaging the linear pattern of labels on the stretched labeled genomic region 18. As mentioned above, the stretched labeled genomic region may be imaged by employing various embodiments of microscopy described above, or by scanning during or after the stretching step 4. If the label is fluorescent, the presence of the label may be detected by the human eye, a camera, flow cytometry, or scanning fluorescence detectors, or a spectrometer, etc. If the nucleotide label is a tag composed of synthetic compounds, nucleic acids, amino acids, or a combination of both nucleic acids and amino acids, prior to reading step 6, the genomic region may be processed to visualize the tag via binding to an epitope presented on the tag, primer extensions, sequencing, or additional processing to identify and locate the label, for example.

The labeling pattern in the form of a test pattern obtained from reading step 6 may then be analyzed by a human or a computer programmed to analyze or compare labeling patterns in the forms of test patterns. In some embodiments, the test pattern is derived by recording a sequence of colors that are incorporated at a plurality of sites in order of their positions along a length of a genomic region. The distance between any pair of labels may also be recorded. The type of methods (ZFPs, oligo hybridization, etc) employed to label the genomic region may also be incorporated into the test pattern. These data recorded for the genomic region under analysis represents a pattern that represents the region of the genomic region into which the labels are incorporated. In certain cases, test patterns with their corresponding images of a fluorescent labeling pattern may be recorded in forms of images or tables correlating emission wavelengths over genomic region length. The test pattern representing the labeling pattern may also be stored as values of emission wavelength at each location along the genomic region length. Other ways of representing and storing test patterns are also envisioned.

The subject method involves utilizing the test pattern to identify the genomic context for a genomic region of interest. In certain cases, the test pattern may be compared as in step 8 to a database of reference patterns derived from a reference genome that has been labeled in the same way as the genomic sample of interest in an experiment or in silico. If the pattern is found to be the same as one that is identified by the reference, the genomic region under study is identified to be the same as that of the reference, effectively mapping the region of the test genome, as shown in step 10 in FIG. 1. For example, if the pattern is RGGRGB (e.g. red, green, green, red, green, blue) and the human chromosome 10, cytoband q10 also has the same pattern, the genomic region under study is identified to be cytoband q10 of chromosome 10. Distance between labels may also be incorporated into the pattern to increase the specificity of the pattern for each identified region. For example, the distances that can be translated into a form of test pattern may include long, short, and/or medium, e.g. L, S, and/or M, respectively. An exemplary test pattern that incorporates distances and colors may look like: RLGLGSR. The number of distances and colors conveyed by the test pattern would depend on how the test genome is labeled and the amount of information required to uniquely identify the genomic region of interest.

These data recorded as a test pattern represents the region of the genomic region into which the labels are incorporated. If the data comprises only two colors (e.g. red (R) and green (G)), or two distances (e.g. long (L) and short (S)), the pattern is considered to be binary. In a binary format, if the pattern has 2 bits, there are 2²=4 unique patterns. E.g., RR, GG, RG, and GR or LL, LS, SL, and SS. The pattern may have 10 bits, providing for 2¹⁰=1024 unique patterns. Accordingly, depending on the number of colors and distances in the pattern, the number of discrete units of information in a pattern may be designed so that each region in a genome may be uniquely identified. For example, if a genome of about 245 million base pairs is divided up into regions of about 10 kb to 100 kb in length, each requiring a unique identifier, there would be about 2,450 to about 24,500 regions. Where the subject method employs a binary pattern system, a 12 to 15 bit-pattern allows for 4,096 to 32,768 unique identifiers. As such, a 12 to 15 bit-pattern may adequately cover the whole genome although bit-patterns beyond 15 bits are also envisioned herein.

Where the pattern comprises more than 2 colors and/or distances between colors, the pattern is then higher in complexity than the binary pattern so the amount of information units required to generate the same number of unique identifiers would be lower. For example, if the pattern contains 3 colors, an 8 to 10 trit-pattern would provide 6,561 to 59,049 unique identifiers. If the pattern contains 4 colors or 2 colors and 2 distances, a 6 to 8 unit-pattern would provide 4,096 to 65,536 unique identifiers, etc. Accordingly, for example, the pattern may be binary (e.g. RGRGRG), ternary (e.g. RGBRGBBRG), quaternary (e.g. RLGSRLGSR), in which each unit of the pattern may be a color or distance. In light of what has been described, various other coding systems may be designed accommodate the various means of labeling genomic DNA or vice versa.

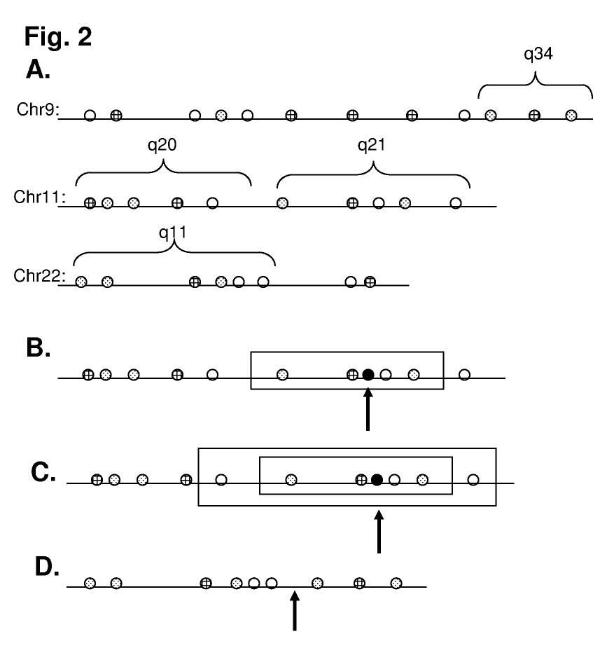

How the test patterns may be used to assign a genomic context to a region and thereby effectively mapping the region is illustrated in FIG. 2. Reference genomes may be labeled experimentally or in silico as illustrated in FIG. 2A. A region in each of chromosome 9, 11, and 22 is labeled site-specifically with three different labels: open circle (O), criss-cross circle (C), and dotted circles (D). Due to differences in nucleotide sequence and/or labeling means employed, different linear patterns are generated for each chromosomal region. As noted above, different coding and labeling systems may be designed to provide a plurality of distinct patterns for a desired coverage of a genome of interest. In the example shown in FIG. 2A, these different patterns allow genomic regions in chromosome 9 to be distinguished from another region in chromosome 11, for example.

Depending on the amount of reference patterns and the amount of labeling information conveyed by the test patterns, different amount of discrete unit of information would be required in a test pattern to successfully map a region of interest. An example is presented in FIG. 2B to describe in detail of how the amount of information unit required in a pattern to successfully map a genomic region may vary depending on the reference patterns and the labeling information conveyed by the test pattern. First, the region of interest in FIG. 2B is labeled in the same way as those in FIG. 2A. Generally, if the linear pattern of the test genome matches a pattern seen in a reference genome, the region in the test genome is identified to be the same as that in the reference genome. Reading from left to right, the leftmost genomic region shown in FIG. 2B has a criss-cross circle (C), and two dotted circles (D). A test pattern representing this leftmost segment of the region would be CDD. Browsing through the reference patterns provided by the linear patterns in FIG. 2A would lead one to discover that there is no CDD in any chromosome other than a region (e.g. q20) in chromosome 11. As such, CDD as a 3-unit pattern is capable of identifying the chromosome number of test genome in FIG. 2B as chromosome 11. On the other hand, if only a 2-unit pattern is used, e.g. CD or DD, a unique chromosome number could not be assigned because CD exists in all three reference regions presented in FIG. 2A and DD exists in both chromosomes 11 and 22.

In addition to assigning a chromosome number, in some embodiments, the genomic context also maps a region of a test genome to a specific region in a chromosome. In some embodiments, a specific region is identified to indicate where a feature of interest resides with a higher resolution than merely providing a chromosome number. Depending on the desired resolution, various sizes of a genomic region may be mapped (e.g. 1 kb, 10 kb, 1000 kb up to 10 MB or more). In FIG. 2B, the region has an additional label illustrated as a filled circle (F) as a feature of interest. This additional label represents a feature of interest, such as state of methylation or acetylation, or a specific nucleotide sequence. For example, a labeled protein that specifically binds to methylated nucleotides may be used to label methylated nucleotides as a feature of interest. To identify the region in which this feature resides, one could use a test pattern representing the labels adjacent to the feature. One such test pattern encompassing the labels adjacent to the feature are derived from the linear pattern boxed in FIG. 2B. This test pattern comprises DCOD, with a filled circled (F) in between C and O. A test pattern of DCOD is found to be adequate in mapping the region as q21 of chromosome 11 shown in FIG. 2A. If the test pattern is shorter than a 4 unit-pattern, e.g. DCO instead of DCOD, a unique region in chromosome 11 would not be able to be identified since there are at two repeats of DCO, one at the end of q20 and another in the beginning of q21. On the other hand, a longer pattern, e.g. DCODO instead of DCOD, would not be necessary since DCOD is adequate in identifying a unique region in chromosome 11. As such, in this embodiment illustrated in FIG. 2B, a conclusion may be drawn that the feature of interest (e.g. methylated nucleotide) resides in q21 of chromosome 11.

Based on the description above, a test pattern that uniquely identifies a region of a test genome is not required to convey information on an entire stretched region of a test genome. Depending on the reference patterns and the type of information conveyed (e.g. colors and/or distances), the test pattern that is capable of identifying a unique genomic context may comprise label information across only a proportion of a contiguous length across the stretched genomic region. The linear pattern on a stretched genomic region that provides the test pattern may be derived from a contiguous stretch of DNA that is at least 1, 10, 50, 100, 500, 1000, or more kb up to a whole intact chromosome. Accordingly, a test pattern the unique identify a region of a test genome may comprise label information across less than 100%, 90%, 80%, 60%, 50%, 30%, 20%, or 10% or less of the contiguous length of the entire stretched region of the test genome. In certain embodiments, the test pattern is not derived from only one labeled site but from at least two, at least three, at least four, up to at least five or more labeled sites in the order of their positions along a contiguous stretch of genomic DNA. These labeled sites are also spaced apart from each other at a distance near or above the diffraction limit for visible wavelengths of light, as described above.

Since the amount of test pattern unit required may vary, reading labels along a region of a test genome may be performed incrementally in certain cases to incorporate additional label information only as needed. For example, if a four unit pattern as boxed in FIG. 2B is not adequate to identify a unique region in the reference patterns, the reading step in the subject method would continue to read one or more additional labels in an order of positions along the length of the stretched genomic region as boxed in the larger box in FIG. 2C. This expansion of test pattern in an incremental fashion may involve comparison with reference patterns with each incrementally expanded text pattern until a unique region is matched. A feedback control may be implemented such that once a unique region is assigned to the region of the test genome, the step of reading the linear pattern in that genomic region may be halted. If no unique reference pattern has been found to match the test pattern, reading the linear pattern is continued to expand the test pattern to enough units of information until a unique region can be assigned.

In addition to mapping a region of interest in the test genome, the subject method may also identify sequence alterations, such as chromosomal translocation. A chromosomal translocation is exemplified in FIG. 2D. From left to right, the test pattern may be DDCD . . . , which when compared with the references shown in FIG. 2A, identifies q11 of chromosome 22 as the region of interest. However, as the test pattern is read to extend to the entire linear pattern, a test pattern of DDCDOODCD does not match with the rest of chromosome 22. Rather, part of the test pattern that represents the right end of the region shown, DCD, is found to match the reference pattern representing q34 of chromosome 9. Accordingly, when one test pattern such as that found in FIG. 2D is found to match two reference patterns, the test pattern indicates a chromosomal translocation, provided that the two reference patterns are derived from discontiguous segments of a reference genome. In the example illustrated in FIG. 2D, the arrow points to the chromosomal breakpoint which is indicated by where the test pattern would cease to match a reference pattern representing q11 of chromosome 22 or a region continuous therewith and begins to match a reference pattern representing q34 of chromosome 9. In a similar vein, many other chromosomal alterations in a range between 10 kb and up to a whole chromosome, including insertions, duplication, deletion, or inversion, for example, may be determined using the subject method.

For mapping a region of interest in the test genome, an algorithm containing instructions for executing the algorithms may also be provided. The algorithm may automate pattern matching between test patterns and reference patterns. The algorithm may enable gaps when aligning test patterns with reference patterns in a similar way as BLAST (Altschul S F et al. (1990) J Mol Biol. 215:403-10) does. The gap may be resulted from a deletion event or incomplete labeling. The algorithm may also calculate the probability at which the gap found in the test genome would exist in the reference genome. In certain cases, a small gap may be caused by incomplete labeling or a short sequence alteration while a large gap may be caused by a deletion or other chromosomal rearrangements. In certain embodiments, the algorithm can determine if a particular sequence is present or absent in a test sample and an instruction may be provided to display the results of that determination. For example, if a particular reference pattern is not found anywhere in the test sample, the sequence represented by the reference pattern may be absent in the test genome. A display then may inform the operator that there is a deletion or other chromosomal rearrangements relative to the reference genome. In certain cases, a deletion or chromosomal rearrangement that may be detected involves sequences in a variety of ranges, from about 1000 bases up to an entire chromosome.

The algorithm and/or instructions to apply the algorithm in the subject method may be provided in a physical storage or transmission medium. A computer receiving the instructions may then execute the algorithm and/or process data obtained from the subject method. Examples of storage media that is computer-readable include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer on a local or remote network.

As noted previously, the subject method involves the analysis of a test genome in a genomic sample. The genomic DNA may undergo staining, shearing, fragmentations by sonication or nebulization (e.g. into fragments between 10 kb to 10,000 kb in size), purification, etc., prior to being labeled site-specifically in the method. In certain embodiments, the test genome to be labeled is at least 50, 100, 500, 1000, or more kb up to a whole intact chromosome in length. As mentioned above, the linear pattern on a stretched genomic region may be derived from a contiguous stretch of DNA that is at least 50, 100, 500, 1000, or more kb up to a whole intact chromosome.

In certain cases, a partitioned genome may be site specifically labeled and analyzed as a test genome in accordance with the subject method. In certain cases, a test genome may be fragmented as described above and a portion of the genome may be isolated, labeled, and analyzed using the subject method. The portion may be a number of chromosomes less than the total number of chromosomes found in the genome of an organism (e.g. one or two chromosomes). The portion may be less than 90%, (e.g. less than 80%, less than 60%, less than 50%, less than 40%, less than 20%, less than 10% or less) of the length of the total genome of an organism. Methods for partitioning genome are known in the art.

Where the agent used to site-specifically label is an enzyme, the enzymes that are employed by the labeling step may be of a bacterial system, of a mammalian origin or a hybrid of various origins. Recognition sequences and protein sequences of many bacterial or mammalian enzymes commonly used in labeling nucleic acids are known and deposited in databases such as the NCBI's GenBank database.

As discussed above, the labeling step incorporates at least two labels that are distinguishable, e.g. different colors that can be distinguished. The label may comprise a detectable component that can be either directly visualized or be processed for indirect visualization. Detectable labels are known in the art and need not described in detail herein. Briefly, exemplary detectable components include radioactive isotopes, fluorophores, fluorescence quenchers, affinity tags, e.g. biotin, crosslinking agents, chromophores, beads, quantum dots, etc. In certain embodiments, the detectable label, such as biotin, may require incubation with a recognition element, such as streptavidin, or with secondary antibodies to yield detectable signals. In other embodiments, the detectable label, such as a fluorophore, may be detected directly without performing additional steps. Additional fluorescent dyes of interest include: xanthene dyes, e.g. fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc. (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPRO3 TOPRO3 (Molecular Probes, Eugene, Oreg.). Further suitable distinguishable detectable labels may be found in Kricka et al. (Ann Clin Biochem. 39:114-29, 2002).

In certain cases, the genomic region under study is stained with a nonspecific label, such as an intercalating fluorescent dye or other dyes that would label DNA in a non-sequence specific manner (e.g. DAPI, Hoechst, YOYO-1, YO-PRO-1, or PicoGreen). In related embodiments, the labels incorporated into the genomic in the site-specifically labeling step may participate in fluorescence energy transfer (FRET) with an adjacent labeled site or with a non-specifically incorporated label (e.g. DNA backbone). The FRET signal is then imaged the same way as the embodiments described above to generate a linear pattern of labeled sites in order of positions along the length of the stretched genomic region.

In carrying out the analysis of a test pattern of the labeled stretched genomic region, a reference image or pattern derived from a reference genome may be used. A reference sequence may be a sequence derived from an identified source or from the same species as the genomic sample under study. The source may be known to be homozygous or heterozygous for a particular genomic locus of interest. In certain cases, the source may be wild-type for a genomic locus of interest. The source may contain an allelic variant of interest. In certain cases, the reference sequence may be known so that the specific nucleotide sequences implicated in a genomic feature of interest (e.g. single nucleotide polymorphism, restriction fragment length polymorphism, genetic mutations, etc.) are known. The reference sequence may also undergo the subject method so that it is labeled in the same way as the genomic sample under interest. In other embodiments, the reference image or reference pattern may be derived in silico based on the information available about the reference sequence, such as those stored in databases. For example, the pattern of labeling may be predicted based on sequence data and type of site-specific labels used.

The present disclosure also provides a system for sample analysis comprising: a) reagents to site-specifically label a test genome with at least two different labels; b) a stretching device; c) an imaging workstation; d) a computer for recording; and e) a computer-readable medium comprising a database of reference patterns. The system may comprise agents such as enzymes, proteins, antibodies, oligonucleotides, small molecules as labeling means described above. The labeling means to be used in the system is also provided to allow incorporation of two different labels in the test genome. The stretching device and imaging work station encompass any instrument employed for the various stretching and imaging means described previously.

The system may include a computer programmed to record and store labeling pattern and may also be programmed to convert the linear labeling pattern into test patterns. The system may encompasse a storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer on a local or remote network. Similarly, a database of reference pattern may also be provided in a computer readable medium in the subject system.

### Kits

Also provided by the present disclosure are kits for practicing the subject method, as described above. The subject kit contains agents such as enzymes, antibodies, oligonucleotide composition, small molecules and/or any other reagents for incorporating at least two different labels into the test genome. The kit may further contain a reference genome or information relating to a reference genome.

In additional embodiments, the reagents included in the kit may allow for more than one labeling means. Specific combinations of labels or labeling means may be designed using the kit in accordance with individual needs.

The kits may be identified by the type of labeling means, the recognition sequence of any agents (e.g. enzymes, binding proteins, oligonucleotides, or small molecules) and/or the reference genome. The kits may be further identified by the method of stretching the labeled region of a test genome and the appropriate coding system. The kits may also include information relating to one or more features of interest that may be identified in the test genome, such as a disease-related nucleotide sequence or nucleotide methylation state.

In addition to above-mentioned components, the subject kit typically further includes instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the instructions, the kits may also include one or more control analyte mixtures, e.g., two or more control analytes for use in testing the kit. In addition to above-mentioned components, the subject kit may include software to perform comparison of the pattern to one or more reference patterns.

### Utility

The subject method finds use in a variety of applications, where such applications are generally nucleic acid detection applications in which the presence of a particular nucleotide sequence in a given sample is detected at least qualitatively, if not quantitatively. In general, the above-described method may be used in order to map a region in a genome based on the generated labeling pattern.

Since contacting step 2 is sequence dependent, the presence or absence of labeling in specific locations in a genomic region is informative of the sequence information in those locations. By comparing the pattern of the labeled genomic region to that of a reference sequence, the genomic context and the identity of the labeled region may be determined.

As noted above, the method provides analysis on a single molecule level, using methods such as those involving microscopy or microfluidic/nanofluidic channels. In particular embodiments, the genomic regions of interest are subjected to DNA stretching or confinement elongation prior to the imaging step. The subject method may also comprise recording the imaged linear pattern as a test pattern comprising a sequence of colors and/or distance between colors. The color represents fluorescence emission of the labeled nucleotides incorporated into the stretched genomic region. This recorded pattern may be used to compare with reference patterns to identify the genomic context and the identity of the labeled region (e.g. chromosome 9, region q34) as described previously. The genomic context that may be assigned to a labeled DNA identifies a segment of the DNA on a scale of about 50, about 100, about 500, up to about 1000 Kb or more. In certain embodiments, the comparison between the recorded pattern and the reference pattern may also determine if there are chromosomal rearrangements or other sequence differences relative to the reference. Sequence alterations (e.g. chromosomal rearrangements) that may be detected include translocations, inversions, tandem duplications, insertions, deletions, SNPs, and other sequence mutations. Chromosomal rearrangements that may be detected by the subject method encompass differences between the test sample and the reference that range from 10 kb fragments, to entire chromosome arm or an entire chromosome, such as a missing chromosome or a chromosome arm duplication, for example.

Analysis carried out using the method may be applied on a genomic scale that involves shearing, fragmenting, amplifying, or processing the genomic DNA in other ways prior to site-specifically labeling the test genome. Although genomic sample may be complex, the pattern generated by the labeling patterns may be designed to be unique for the region of genome under study. Many labeling patterns may be generated in accordance with the many embodiments of the method described above so as to provide unique patterns for each of a plurality of genomic regions. As mentioned above, each genomic region identified may be on a scale of about 50, about 100, about 500, about 1000 Kb, up to about 10 Mb or more in length.

Other assays of interest which may be practiced using the subject method include: genotyping, scanning of known and unknown mutations, gene discovery assays, genomic structural mapping, differential gene expression analysis assays, nucleic acid sequencing assays, and the like.

The pattern measured through the use of the subject methods can also be compared to a set of several reference patterns with the purpose of identifying the closest one. This might represent comparison between sequences coming from variants of a region or of an entire genome. Identification of the pattern in a sample genome may be useful for a wide variety of investigations, such as identifying origin of a crop, identifying species of fish or other animals, identifying pathogens, or distinguishing between a finite number of known genotypes. For example, a certain pattern in a human genome may identify that one DNA region is translocated or inverted with respect to the reference genome. Analysis of genomic rearrangements is useful in research on certain cancers, for example (De Lellis et al., Ann. Oncol. 18 Supp6: vil73-178 (2007)).

In certain cases, the genomic sample under study may be derived from a sample tissue suspected of a disease or infection. Performing the subject method to analyze the genomic sample from such sample tissues would be useful for disease diagnosis and prognosis. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Aside from providing a genomic context the purpose of mapping a region of a test genome, one or more labeling means may provide additional information relating to feature of interest, such as methylation state, SNP, or a disease-causing nucleotide sequence. Where SNP is a feature of interest, the recognition sequence of an agent (e.g. enzyme, antibody, small molecules or oligonucleotide) used in the labeling step overlaps a site of single nucleotide polymorphism (SNP) in the test genome or reference sequence. Since the nucleotide sequences of hundreds of thousand of SNPs from humans, other mammals (e.g., mice), and a variety of different plants (e.g., corn, rice and soybean), are known (see, e.g., Riva et al 2004, “A SNP-centric database for the investigation of the human genome” BMC Bioinformatics 5:33; McCarthy et al 2000 “The use of single-nucleotide polymorphism maps in pharmacogenomics” Nat Biotechnology 18:505-8) and are available in public databases (e.g., NCBI's online dbSNP database, and the online database of the International HapMap Project; see also Teufel et al 2006 “Current bioinformatics tools in genomic biomedical research” Int. J. Mol. Med. 17:967-73), the labeling of genomic DNA to identify an SNP would be well within the skill of one of skilled in the art. The SNP may be known prior to choosing the labeling means based on the recognition sites of the agent. In certain embodiments, individual SNPs may differ among genomic sample as to destroy or to change certain recognition sequences relative to a human genome reference sequence, and other SNPs may create recognition sequences. Therefore, individual DNA samples may have different labeling patterns than that of a reference after being subjected to the method provided herein.

The above described applications are merely representations of the numerous different applications for which the subject array and method of use are suited. In certain embodiments, the subject method includes a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

### EXAMPLE

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Model

The (human) genome is tagged in-silico using the following two recognition sequences (and their watson-crick complements): ‘GCTCTTC’ and ‘CGAGAAG’ The experiment was carried out in accordance with the following steps.

Step 1

This in-silico tagging incorporates information petaining to the expected optical resolution of the measurement. Consecutive tags of the same color (e.g. R or G) were further annotated as having an unclear resolvability status if they are too close in distance to each other. An unclear resolvability status denotes possibly a optical merging of the two tags. The annotation was presented by x between the two tags in this example pattern: “ . . . RRGxGGGRGRxRxRRGG . . . ”. An x between two tags denoted a distance between the loci for the two tags that is shorter than the prevailing resolution (RES).

Step 2

Random fragments of length L were drawn in the genome. The start positions of these fragments were uniformly distributed.

Step 3

A pattern of tags was generated for each randomly drawn fragment. This pattern was binary sequence over the letters R and G for example, in this case, to represent two color-labeling. When two or more tags of the same color was observed as consecutive occurrences and the distance between the two tags was lower that the prevailing resolution, RES, the decision of whether a single letter or two letters represent the pair in the pattern was made by tossing a p=0.5 coin. Thus, if the pattern observed in the genome, at the randomly drawn locus, was
GGRxRRRxRGGRRGxGR,
   then the generated pattern would be either

a. GGRRRGGRRGR with a probability of 0.125

b. GGRRRGGRRGGR with a probability of 0.125

c. GGRRRRGGRRGR with a probability of 0.25

d. GGRRRRGGRRGGR with a probability of 0.25

e. GGRRRRRGGRRGR with a probability of 0.125

f. GGRRRRRGGRRGGR with a probability of 0.125

Step 4

Consider a given genomic pattern, T, over R, G and x, generated according the rules described in Step 1. The potential observables of T, denoted OBS(T), were defined as the set of all patterns over R and G that can result from T according to Step 3

Step 5

The pattern generated in Step 3, denote by Q, was used as a query pattern to search in the genome pattern of tags as generated in Step 1.

Step 6

A genomic locus was considered a potential hit for Q if and only if the pattern of tags, T, corresponding to the genomic locus satisfied the condition that Q is a member of OBS(T).

Step 7

For the simulations study, the random drawing was performed 1000 times, for various values of L and of RES, and the following frequencies were recorded as metrics of the system performance:
a. Unique hits—frequency of random fragments that have a unique hit. This fraction represents an ability to map an observed fragment to a unique position in the genome.
b. 2-5 hits—frequency of random fragments for which 2-5 hits are found, according to Step 6 above. This fraction represents fragments that can be mapped to a small number of loci.
c. >5 hits—frequency of random fragments for which more than 5 hits are found, according to Step 6 above. This fraction represents fragments that are mapped to a large number of genomic loci.
d. Un-tagged—frequency of random fragments that contain none of the two tagged sequences and therefore do not give rise to any pattern. This fraction represents fragments that can not be mapped back to the genome, using the settings of this model.

Results:

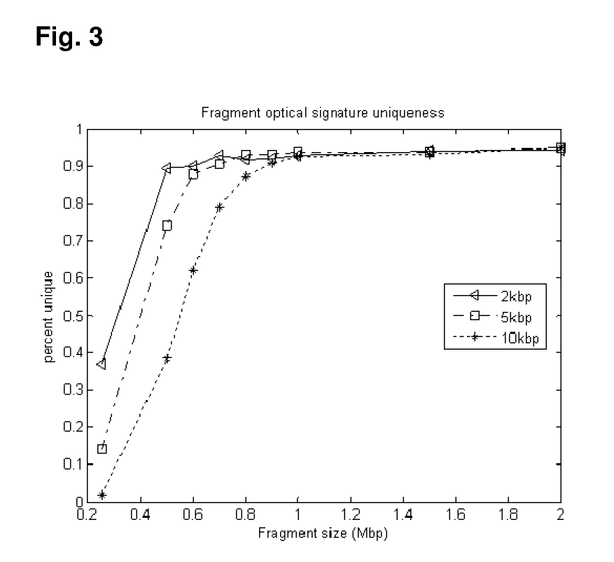

The above simulations were performed with various values for L (the length of the random fragments) and RES (the prevailing assumed optical resolution). The results are summarized in FIG. 3 and the tables below. The results presented here are based on drawing 1000 random fragments for each pair of values L and RES.

TABLE 1
Percentage of uniquely mappable fragments. Results of Table 1 are presented in FIG. 3

| | Frag size (L); Mbp | | | | | | | | |
|---|---|---|---|---|---|---|---|---|---|
| | .25 | 0.5 | 0.6 | 0.7 | 0.8 | 0.9 | 1 | 1.5 | 2 |
| 2 kbp | 0.3700 | 0.8940 | 0.9000 | 0.9270 | 0.9170 | 0.9190 | 0.9280 | 0.9400 | 0.9420 |
| res | | | | | | | | | |
| 5 kbp | 0.1420 | 0.7420 | 0.8790 | 0.9040 | 0.9320 | 0.9320 | 0.9370 | 0.9380 | 0.9490 |
| res | | | | | | | | | |
| 10 kbp | 0.0190 | 0.3850 | 0.6220 | 0.7900 | 0.8740 | 0.9090 | 0.9250 | 0.9300 | 0.9480 |
| res | | | | | | | | | |

TABLE 2
Percentage of fragments that each has 2, 3, or 4 hits.

| | Frag size (L); Mbp | | | | | | | | |
|---|---|---|---|---|---|---|---|---|---|
| | .25 | 0.5 | 0.6 | 0.7 | 0.8 | 0.9 | 1 | 1.5 | 2 |
| 2 kbp | 0.1300 | 0.0320 | 0.0140 | 0.0030 | 0.0040 | 0.0020 | 0.0030 | 0 | 0 |
| res | | | | | | | | | |
| 5 kbp | 0.1000 | 0.1190 | 0.0400 | 0.0340 | 0.0060 | 0.0010 | 0 | 0.0010 | 0.0020 |
| res | | | | | | | | | |
| 10 kbp | 0.0300 | 0.2010 | 0.1650 | 0.1040 | 0.0470 | 0.0250 | 0.0100 | 0.0020 | 0 |
| res | | | | | | | | | |

TABLE 3
Percentage of fragments that each has 5 or more hits.

| | Frag size (L); Mbp | | | | | | | | |
|---|---|---|---|---|---|---|---|---|---|
| | .25 | 0.5 | 0.6 | 0.7 | 0.8 | 0.9 | 1 | 1.5 | 2 |
| 2 kbp | 0.4250 | 0.0160 | 0.0070 | 0.0100 | 0.0050 | 0.0080 | 0.0020 | 0.0030 | 0.0030 |
| res | | | | | | | | | |
| 5 kbp | 0.6820 | 0.0720 | 0.0200 | 0.0060 | 0.0070 | 0.0030 | 0.0040 | 0.0090 | 0.0080 |
| res | | | | | | | | | |
| 10 kbp | 0.8920 | 0.3480 | 0.1500 | 0.0530 | 0.0170 | 0.0170 | 0.0050 | 0.0020 | 0.0070 |
| res | | | | | | | | | |

TABLE 4
Percentage of fragments that are untagged.

| | Frag size (L); Mbp | | | | | | | | |
|---|---|---|---|---|---|---|---|---|---|
| | .25 | 0.5 | 0.6 | 0.7 | 0.8 | 0.9 | 1 | 1.5 | 2 |
| 2 kbp | 0.0750 | 0.0580 | 0.0790 | 0.0600 | 0.0740 | 0.0490 | 0.0670 | 0.0570 | 0.0550 |
| res | | | | | | | | | |
| 5 kbp | 0.0760 | 0.0670 | 0.0610 | 0.0560 | 0.0550 | 0.0640 | 0.0590 | 0.0520 | 0.0410 |
| res | | | | | | | | | |
| 10 kbp | 0.0590 | 0.0660 | 0.0630 | 0.0530 | 0.0620 | 0.0710 | 0.0600 | 0.0660 | 0.0450 |
| res | | | | | | | | | |

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of sample analysis comprising:
a) site-specifically labeling a test genome with at least two different labels to produce a labeled genome labeled at a plurality of discrete sites across said genome;
b) stretching a nucleic acid of said labeled genome to produce a linear pattern of said different labels along a region of a stretched nucleic acid;
c) reading said labels along said region to provide a test pattern comprising a sequence of colors emitted by said labels;
d) comparing said test pattern to a plurality of reference patterns obtained from a reference genome, wherein said reference patterns are mapped to corresponding genomic locations in said reference genome; and
e) identifying one or more reference patterns that match said test pattern, thereby mapping a location for said region in said test genome.

2. The method of claim 1, wherein said reading step c) comprises evaluating a distance between said labels in said stretched nucleic acid to provide a pattern comprising a sequence of said colors and said distance between said labels.

3. The method of claim 1, wherein said reading step c) comprises reading labels in order of their positions along a length of said region that is less than the total length of said labeled genome.

4. The method of claim 1, wherein step a) comprises labeling said test genome with three or more different labels, and wherein reading step c) comprises reading said labels along said region to provide a test pattern comprising a sequence of three or more different colors emitted by said labels.

5. The method of claim 1, wherein said identifying step e) comprises identifying two or more reference patterns that match said test pattern, thereby indicating that said region comprises a chromosomal rearrangement relative to said reference genome.

6. The method of claim 5, further comprising: identifying a chromosomal break point within said region of said stretched nucleic acid.

7. The method claim 1, further comprising labeling said genome with a third label.

8. The method of claim 7, wherein said third label is incorporated into said genome by an agent that is specific for a recognition site of interest.

9. The method of claim 8, wherein said recognition site comprises a methylated nucleotide.

10. The method of claim 1, wherein step a) comprises labeling one or more transcription factor binding sites.

11. The method of claim 1, wherein step a) comprises contacting said test genome with a labeled oligonucleotide.

12. The method of claim 1, wherein step a) comprises contacting said test genome with an antibody.

13. The method of claim 1, wherein said stretching step b) comprises stretching said nucleic acid in a nanochannel.

14. The method of claim 1, wherein said region of said stretched nucleic acid read to provide said test code is at least about 100 kilobases in length.

15. The method of claim 1, wherein said linear pattern of said different labels along said region of said stretched nucleic acid comprises a density of label less than one label for every 1000 base pairs.

16. The method of claim 1, wherein said step c) provides a plurality of test patterns, each of which corresponds to a different region of said stretched nucleic acid.

17. The method of claim 15, wherein said identifying step e) comprises identifying one or more reference patterns for each of said plurality of test patterns to provide a haplotype for said stretched nucleic acid.

18. The method of claim 1, wherein said reference genome is from the same species as that of said test genome.

19. A system comprising:
a) reagents for site-specifically labeling a test genome with at least two different labels to produce a labeled genome labeled at a plurality of discrete sites across said genome;
b) a stretching device;
c) an imaging workstation;
d) a computer for recording a pattern; and
e) a readable computer medium comprising a database of reference patterns.

20. The system of claim 19, wherein said stretching device comprises a nanochannel.
